(19) United States
(12) Patent Application Publication
(10) Pub. No.: US 2011/0086436 A1
(43) Pub. Date: Apr. 14, 2011

(54) METHOD FOR DETECTING ANTIGEN, AND APPARATUS FOR DETECTING ANTIGEN USING THE SAME, AND MICROFLUIDIC CHIP USING THE SAME

(75) Inventor: Nam Soo MYUNG, Seongnam (KR)

(73) Assignee: Electronics and Telecommunications Research Institute, Daejeon (KR)

(21) Appl. No.: 12/900,316

(22) Filed: Oct. 7, 2010

(30) Foreign Application Priority Data
Oct. 9, 2009 (KR) 10-2009-0096447
Aug. 27, 2010 (KR) 10-2010-0083629

(51) Int. Cl. G01N 33/566; C40B 60/12; G01N 30/00

(52) U.S. Cl. 436/501; 506/39; 422/69

(57) ABSTRACT

A method for detecting an antigen, an apparatus for detecting an antigen using the same, and a microfluidic chip using the same are disclosed. The method for detecting an antigen, includes: binding a first antibody and nano-beads to generate antibody nano-beads; binding the generated antibody nano-beads and an antigen to generate antigen-antibody nano-beads; forming at least one of an electric field and a magnetic field on the generated antigen-antibody nano-beads to bind the generated antigen-antibody nano-beads and a second antibody; and detecting the antigen-antibody nano-beads bound to the second antibody. Thus, when nano-beads affected by an electromagnetic field exist within the electromagnetic field that temporally and spatially changes, the nano-beads move according to non-uniformity of the electromagnetic field. In particular, an active mixing can be performed by using the electromagnetic field which is spatially non-uniform and changes temporally, a reaction time can be reduced. In addition, a flow can be controlled to make nano-beads move to a capture antibody, thus detecting an antigen with a small amount of a test sample within a short time.

### CROSS-REFERENCE TO RELATED APPLICATIONS

This application claims the priority of Korean Patent Application No. 10-2009-0096447 filed on Oct. 9, 2009 and Korean Patent Application No. 10-2010-0083629 filed on Aug. 27, 2010, in the Korean Intellectual Property Office, the disclosure of which is incorporated herein by reference.

### BACKGROUND OF THE INVENTION

1. Field of the Invention

The present invention relates to a method for detecting an antigen, an apparatus for detecting an antigen using the same, and a microfluidic chip using the same, and more particularly, to a method for detecting an antigen capable of effectively detecting an antigen by forming an electromagnetic field which is spatially non-uniform and changes temporally, an apparatus for detecting an antigen using, the same, and a microfluidic chip using the same.

2. Description of the Related Art

A general method for detecting the presence or absence of a particular antigen in a test sample will now be described. First, fluorescent antibody nano-beads formed by attaching an antibody which selectively binds to a particular antigen to nano-beads having a fluorescent component is prepared. The fluorescent antibody nano-beads are mixed with a test sample in use for testing the presence or absence of a particular antigen in a certain ratio, and when the particular antigen and the antibody are reacted with each other, fluorescent antigen-antibody nano-beads in which the antibody and the antigen are combined are produced. As a result, the detection of the fluorescent antigen-antibody nano-beads implies the presence of the particular antigen in the test sample.

In general, in a bio-sensor using microfluidics, a small amount of a test sample is transferred to a mixing chamber through a portion in which the fluorescent antibody nano-beads are contained.

After passing through the mixing chamber, the fluorescent antigen-antibody nano-beads pass through a detection chamber on a flow path of a fluid. The detection chamber includes an area where an antigen is attached to a wall surface of a fluid channel.

While the fluorescent antigen-antibody nano-beads, in which the antigen binds to the antibody attached to the surface of fluorescent nano-beads, are passing through the detection chamber, the antibody captured by the wall of the flow path of the fluid and the antigen present on the surface of the fluorescent antigen-antibody nano-beads bind to each other to attach the fluorescent antigen-antibody nano-beads to a surface of the detection chamber.

After a sufficient reaction time has lapsed, non-reacted antigen nano-beads (which have not been attached to the antibody captured by the surface of the flow path) are washed out of the detection chamber. When light is irradiated to the detection chamber from the exterior, fluorescence is generated from the fluorescent antibody nano-beads fixed through the selective antigen and antibody.

Namely, through the detection of such fluorescence, the bio-sensor can detect the antigen by using a small amount of the test sample and measure an antigen concentration in the test sample.

Meanwhile, as the fluorescent antigen-antibody nano-beads are adrift in the form of colloid, making a Brownian movement, in the fluid, natural binding may occur. However, in this case, the fluorescent antigen-antibody nano-beads and the antibody can bind to each other only when they are positioned to be sufficiently close. Namely, a sufficient amount of time is required for the fluorescent antigen-antibody nano-beads to which the antigen binds to react to the antibody captured by the surface of the flow path.

In addition, according to a laminar flow phenomenon in which the fluid flows within the channel, the fluid flow has a zero speed on the wall surface of the channel and a maximum speed at the center of the channel, large colloid particles are largely concentrated to the central portion of the channel and move along with the fluid. Namely, when the fluorescent antigen-antibody nano-beads flow in the channel, they need to be forced to approach the channel wall where the capture antigen is present so as to make the antigen and the antibody bind to each other.

As a result, in the case where the binding of the antibody present on the channel wall and the fluorescent antigen-antibody nano-beads is reliant on Brownian Movement with the fluid stopped, a long period of time is required, and when the fluid is allowed to flow, laminar flow is formed in terms of the characteristics of the fluid flow, causing the fluorescent antigen-antibody nano-beads to concentrate in the central portion of the channel to flow. Namely, the probability that the fluorescent antigen-antibody nano-beads and the capture antibody will react to each other is reduced, requiring a long period of time and a large amount of test samples.

Namely, the related art has the following problems: First, a sufficient amount of time and a sufficient amount of test samples must be provided to allow for a required binding reaction between the antigen within the test sample and the fluorescent antibody nano-beads, and second, a sufficient time and a sufficient amount of test samples must be provided to allow for a required binding reaction between the capture antibody and the antigen-antibody nano-beads.

### SUMMARY OF THE INVENTION

An aspect of the present invention provides a method for detecting an antigen capable of effectively detecting an antigen by forming an electromagnetic field which is spatially non-uniform and changes temporally.

Another aspect of the present invention provides an apparatus for detecting an antigen by forming an electromagnetic field which is spatially non-uniform and changes temporally.

Another aspect of the present invention provides a microfluidic chip using the method for detecting an antigen capable of effectively detecting an antigen by forming an electromagnetic field which is spatially non-uniform and changes temporally.

According to an aspect of the present invention, there is provided a method for detecting an antigen, including: binding a first antibody and nano-beads to generate antibody nano-beads; binding the generated antibody nano-beads and an antigen to generate antigen-antibody nano-beads; forming at least one of an electric field and a magnetic field on the generated antigen-antibody nano-beads to bind the generated antigen-antibody nano-beads and a second antibody; and detecting the antigen-antibody nano-beads bound to the second antibody.

The nano-beads may include at least one of a dielectric and a metal.

The dielectric may have a dipole moment.

The nano-beads may include a fluorescent component.

In generating the antigen-antibody nano-beads, at least one of an electric field and a magnetic field may be formed on the antibody nano-beads.

In binding the generated antigen-antibody nano-beads and the second antibody, at least one of the electric field and the magnetic field may be non-uniform.

In binding the generated antigen-antibody nano-beads and the second antibody, the second antibody may be attached to a fixed position.

According to another aspect of the present invention, there is provided an apparatus for detecting an antigen, including: a mixing chamber for binding antibody nano-beads formed with a first antibody and nano-beads to an antigen to generate antigen-antibody nano-beads; a detection chamber for binding the antigen-antibody nano-beads and a second antibody; and an electromagnetic field generation unit for forming at least one of an electric field and a magnetic field on at least one of the mixing chamber and the detection chamber.

The nano-beads may include at least one of a dielectric and a metal.

The dielectric may have a dipole moment.

The nano-beads may include a fluorescent component.

At least one of the electric field and the magnetic field of the electromagnetic field generation unit may be non-uniform.

The second antibody may be attached to the detection chamber.

According to another aspect of the present invention, there is provided a microfluidic chip including: a mixing chamber for binding antibody nano-beads formed with a first antibody and nano-beads to an antigen to generate antigen-antibody nano-beads; a detection chamber for binding the antigen-antibody nano-beads and a second antibody; and an electromagnetic field generation unit for forming at least one of an electric field and a magnetic field on at least one of the mixing chamber and the detection chamber.

The nano-beads may include at least one of a dielectric and a metal.

The dielectric may have a dipole moment.

The nano-beads may include a fluorescent component.

The second antibody may be attached to the detection chamber.

At least one of the electric field and the magnetic field of the electromagnetic field generation unit may be non-uniform.

The electromagnetic field generation unit may be formed as at least one of an electrode array and a micro-coil array.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a flow chart illustrating the process of a method for detecting an antigen according to an exemplary embodiment of the present invention;

FIG. 2 is a schematic block diagram showing an apparatus for detecting an antigen using an antigen detection method according to an exemplary embodiment of the present invention; and

FIG. 3 is a conceptual view for explaining a microfluidic chip using an antigen detection method according to an exemplary embodiment of the present invention.

### DETAILED DESCRIPTION OF THE PREFERRED EMBODIMENT

The present invention may be modified variably and may have various embodiments, particular examples of which will be illustrated in drawings and described in detail.

However, it should be understood that the following exemplifying description of the invention is not intended to restrict the invention to specific forms of the present invention but rather the present invention is meant to cover all modifications, similarities and alternatives which are included in the spirit and scope of the present invention.

While terms such as “first” and “second,” etc., may be used to describe various components, such components must not be understood as being limited to the above terms. The above terms are used only to distinguish one component from another. For example, a first component may be referred to as a second component without departing from the scope of rights of the present invention, and likewise a second component may be referred to as a first component. The term “and/or” encompasses both combinations of the plurality of related items disclosed and any item from among the plurality of related items disclosed.

When a component is mentioned as being “connected” to or “accessing” another component, this may mean that it is directly connected to or accessing the other component, but it is to be understood that another component may exist there between. On the other hand, when a component is mentioned as being “directly connected” to or “directly accessing” another component, it is to be understood that there are no other components in-between.

The terms used in the present application are merely used to describe particular embodiments, and are not intended to limit the present invention. An expression used in the singular encompasses the expression of the plural, unless it has a clearly different meaning in the context. In the present application, it is to be understood that the terms such as “including” or “having,” etc., are intended to indicate the existence of the features, numbers, operations, actions, components, parts, or combinations thereof disclosed in the specification, and are not intended to preclude the possibility that one or more other features, numbers, operations, actions, components, parts, or combinations thereof may exist or may be added.

Unless otherwise defined, all terms used herein, including technical or scientific terms, have the same meanings as those generally understood by those with ordinary knowledge in the field of art to which the present invention belongs. Such terms as those defined in a generally used dictionary are to be interpreted as having meanings equal to the contextual meanings in the relevant field of art, and are not to be interpreted as having ideal or excessively formal meanings unless clearly defined as having such in the present application.

Embodiments of the present invention will be described below in detail with reference to the accompanying drawings, where those components are rendered using the same reference number that are the same or are in correspondence, regardless of the figure number, and redundant explanations are omitted.

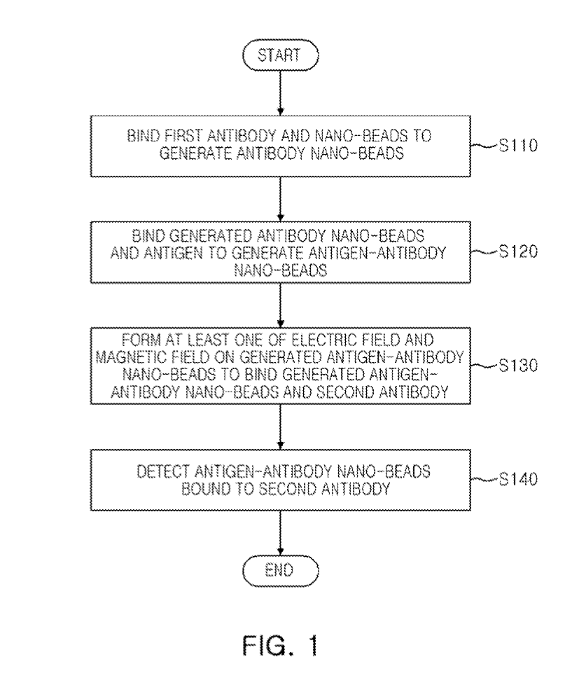

FIG. 1 is a flow chart illustrating the process of a method for detecting an antigen according to an exemplary embodiment of the present invention.

With reference to FIG. 1, a method for detecting an antigen according to an exemplary embodiment of the present invention may include a step S110 of binding a first antibody and nano-beads to generate antibody nano-beads, a step S120 of binding the generated antibody nano-beads and an antigen to generate antigen-antibody nano-beads; a step S130 of forming at least one of an electric field and a magnetic field on the generated antigen-antibody nano-beads to bind the generated antigen-antibody nano-beads and a second antibody, and a step S140 of detecting the antigen-antibody nano-beads that binds to the second antibody.

First, the nano-beads may be configured to include at least one of a dielectric and a metal, and the dielectric may have a dipole moment.

When the nano-beads are configured to include a dielectric or a metal, they receive a force under an electric field and a magnetic field. Thus, the nano-beads move according to a force applied thereto within a non-uniform electric field and magnetic field. Namely, the nano-beads may be controlled to make a desired movement while the electric field and the magnetic field are maintained to be non-uniform.

For example, when the nano-beads are surface-coated with a dielectric or a metal component, the nano-beads receive a force within a non-uniform electric field and a non-uniform magnetic field, and when the non-uniform electric field and the non-uniform magnetic field are controlled, the nano-beads can be controlled to move to a desired position.

In addition, the nano-beads may include a fluorescent component. When the nano-beads contain a fluorescent component, when the presence or absence of an antigen is finally determined, it can be simply processed.

Namely, the presence or absence of an antigen within a test sample can be simply recognized by irradiating light of a wavelength band to which the fluorescent component within the test sample reacts. Namely, when the fluorescent component reacts to the irradiated light, it May be determined that a particular antigen exists in the test sample.

Next, the step S110 of generating antibody nano-beads by binding a first antibody and the nano-beads may be a preparation for determining the presence or absence of the antigen in the test sample. Namely, in step S110, an antibody that can bind to an antigen is prepared in order to determine whether or not an antigen is present.

In addition, in order to easily determine whether or not an antigen is present at a final stage, nano-beads are bound to an antibody so as to be prepared in advance. By using the nano-beads bound to the antibody, the movement of the nano-beads may be forced or the nano-beads may be allowed to assume a fluorescent quality in detecting an antigen.

Next, in step S120 of generating antigen-antibody nano-beads by binding the generated antibody nano-beads and an antigen, the antigen existing in the test sample and antibody nano-beads are bound to determine whether or not an antigen is present in the test sample. Antigen-antibody nano-beads are generated according to the binding.

Namely, if there is no antigen in the test sample, antigen-antibody nano-beads would not be generated, so an antigen cannot be detected in a follow-up step, but if an antigen exists in the test sample, antigen-antibody nano-beads could be generated by the binding and an antigen can be detected in a follow-up step.

In addition, in step S120 of generating the antigen-antibody nano-beads, at least one of an electric field and a magnetic field is formed on the antibody nano-beads. Basically, the generation of the antigen-antibody nano-beads relies on Brownian movement of an antigen and antibody nano-beads. However, the generation of the antigen-antibody nano-beads only relying on Brownian movement is time consuming and requires a large amount of test samples.

Thus, when an electric field and a magnetic field are applied to the antigen and the antibody nano-beads, the antibody nano-beads having a dielectric and metal component can move more actively and the number of generated antigen-antibody nano-beads can be increased according to the more active movement. This is therefore a more positive and effective antigen-antibody nano-beads generation method.

Step S130 of binding the generated antigen-antibody nano-beads and a second antibody by forming at least one of the electric field and the magnetic field on the generated antigen-antibody nano-beads is to determine whether or not an antigen exists in the test sample. This step may be performed to detect nano-beads as a basis for determination.

According to a general method, the antigen-antibody nano-beads are bound to the second antibody while making Brownian movement, and whether or not there is an antigen in the test sample is determined by detecting the nano-beads. However, relying only on Brownian movement has many problems as mentioned above.

Thus, at least one of the electric field and the magnetic field is applied to the antigen-antibody nano-beads to force the antigen-antibody nano-beads to move to a position closer to the second antibody to thus provide more chances to cause a reaction between the antigen-antibody nano-beads and the second antibody.

Here, in step S130 of binding the generated antigen-antibody nano-beads and the second antibody, at least one of the formed electric field and magnetic field may be non-uniform.

If the electric field and the magnetic field are formed to be uniform, the antigen-antibody nano-beads would be simply maintained in an aligned state. Thus, the electric field and magnetic field are formed to be non-uniform so as to force the antigen-antibody nano-beads to move.

The antigen-antibody nano-beads may be forced to move closer to the second antibody. Then, the number of reactions can be increased, which can facilitate the detection of the antigen.

Here, in the step of binding the generated antigen-antibody nano-beads and the second antibody, the second antibody may be attached to a fixed position. In order to easily detect the antigen-antibody nano-beads bound to the second antibody, the position of the antigen-antibody nano-beads must be known. Thus, the second antibody is attached to a fixed position to facilitate the detection of the antigen.

In addition, the force applied to the antigen-antibody nano-beads by applying at least one of the electric field and the magnetic field may be controlled within a range in which the antigen-antibody nano-beads already attached to the second antibody are not detached.

Next, in step S140 of detecting the antigen-antibody nano-beads bound to the second antibody, the antigen-antibody nano-beads may be detected to check whether or not there is an antigen in the test sample.

The antigen-antibody nano-beads can be detected in various manners. In general, fluorescent nano-beads are used as the nano-beads, and in order to detect the antigen-antibody nano-beads, light of a wavelength band to which the fluorescent nano-beads react may be irradiated onto a test sample to cause a fluorescent reaction.

In addition, there may be various detection methods by using the characteristics of nano-beads, and any method can be applicable to the present invention so long as it performs detection by using the characteristics of the nano-beads.

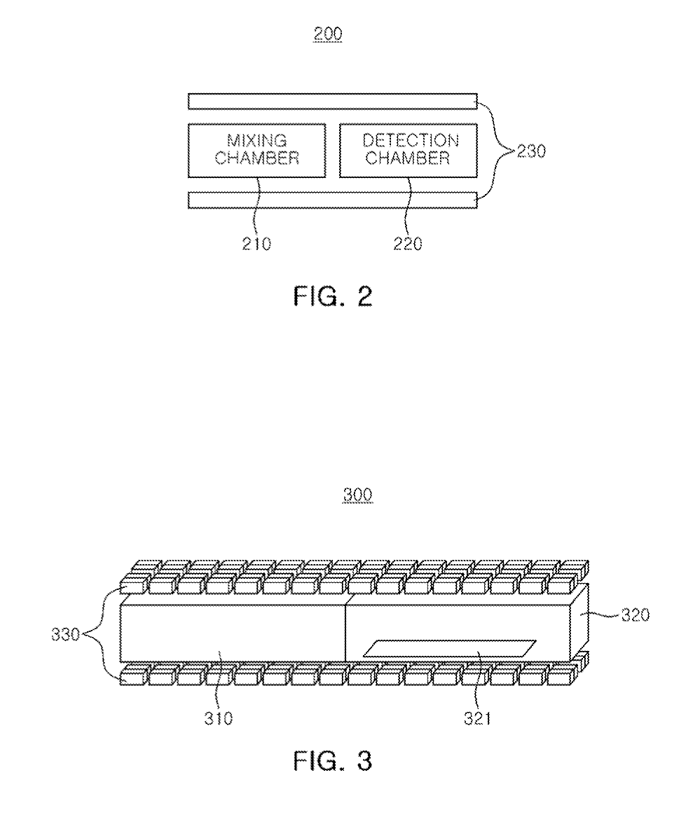

FIG. 2 is a schematic block diagram showing an apparatus for detecting an antigen using an antigen detection method according to an exemplary embodiment of the present invention.

An antigen detection apparatus 200 using the antigen detection method according to an exemplary embodiment of the present invention may be configured to include a mixing chamber 210 for binding antibody nano-beads formed with a first antibody and nano-beads to an antigen to generate antigen-antibody nano-beads, a detection chamber 220 for binding the antigen-antibody nano-beads and a second antibody, and an electromagnetic field generation unit 230 for forming at least one of an electric field and a magnetic field on at least one of the mixing chamber and the detection chamber.

First, the nano-beads used for the antigen detection apparatus 200 may include at least one of a dielectric and a metal. Namely, the surface of the nano-beads may be coated with at least one of a dielectric or a metal.

Because the surface of the nano-beads is coated with at least one of the dielectric and the metal, the nano-beads may received a force within the electric field and the magnetic field by using a dipole moment of the dielectric and also receive a force within the electric field and the magnetic field according to the characteristics of the metal component.

In addition, the nano-beads used for the antigen detection apparatus 200 may include a fluorescent component. Namely, as the nano-beads include the fluorescent component, a method of irradiating light to which the fluorescent component reacts may be used to detect an antigen in a test sample.

The mixing chamber 210 for generating antigen-antibody nano-beads by binding the antibody nano-beads formed with the first antibody and the nano-beads to an antigen may be an area in which a test sample for determining the presence of an antigen and a solution containing the antibody nano-beads are mixed.

In the solution, the antigen and the antibody nano-beads may bind to each other basically by Brownian movement. In addition, more reaction opportunities can be provided to the antigen and the antibody nano-beads by at least one of the electric field and the magnetic field applied by the electromagnetic field generation unit 230 than relying on the general Brownian movement.

In the detection chamber 220 for binding the antigen-antibody nano-beads and the second antibody, a solution containing the antigen-antibody nano-beads may be input thereinto to allow the antigen-antibody nano-beads to bind to the second antibody.

Here, the second antibody may have been attached to the detection chamber. Namely, the antigen-antibody nano-beads may bind to the second antibody attached to the detection chamber according to Brownian movement.

In addition, the antigen-antibody nano-beads may move to a position closer to the second body by at least one of the electric field and the magnetic field applied by the electromagnetic field generation unit 230, and accordingly, the antigen-antibody nano-beads can have more opportunities for binding.

The electromagnetic field generation unit 230, which forms at least one of the electric field and the magnetic field on at least one of the mixing chamber and the detection chamber, may apply a force to the antibody nano-beads in the mixing chamber to induce the antibody nano-beads to be actively mixed with the antigen so as to accelerate the binding to each other, and apply a force to the antigen-antibody nano-beads in the detection chamber to induct the antigen-antibody nano-beads to actively bind to the second antibody.

Here, at least one of the electric field and the magnetic field generated by the electromagnetic field generation unit may be non-uniform. Namely, the antibody nano-beads and the antigen-antibody nano-beads may receive a force according to the non-uniform electric field and magnetic field, and more binding opportunities can be provided according to the movement of the antibody nano-beads and the antigen-antibody nano-beads.

FIG. 3 is a conceptual view for explaining a microfluidic chip using an antigen detection method according to an exemplary embodiment of the present invention.

With reference to FIG. 3, a microfluidic chip using the antigen detection method according to an exemplary embodiment of the present invention may be configured to include a mixing chamber 310 for binding antibody nano-beads formed with a first antibody and nano-beads to an antigen to generate antigen-antibody nano-beads, a detection chamber 320 for binding the antigen-antibody nano-beads and a second antibody, and an electromagnetic field generation unit 330 for forming at least one of an electric field and a magnetic field on at least one of the mixing chamber and the detection chamber.

The mixing chamber 310, the detection chamber 320, and the electromagnetic field generation unit 330 have been described in detail above, so a detailed description thereof will be omitted.

The electromagnetic field generation unit 330 may be formed as at least one of an electrode array and a micro-coil array. This is to generate at least one of the electric field and the magnetic field such that it is non-uniform, and control the electric field and the magnetic field to thus enhance the efficiency of an antigen detection.

In addition, a second antibody attached area 321 is an area on which the second antibody is attached. A force is applied to the antigen-antibody nano-beads to make the antigen-antibody nano-beads to move to the area on which the second antibody is attached, and accordingly, the binding opportunities between the second antibody and the antigen-antibody nano-beads can be increased. Namely, the efficiency of the antigen detection can be improved.

As set forth above, according to exemplary embodiments of the invention, in the method for detecting an antigen, the apparatus for detecting an antigen using the same, and the microfluidic chip using the same, when nano-beads affected by an electromagnetic field exist within the electromagnetic field that temporally and spatially changes, the nano-beads move according to non-uniformity of the electromagnetic field. In particular, an active mixing can be performed by using the electromagnetic field which is spatially non-uniform and changes temporally, a reaction time can be reduced. In addition, a flow can be controlled to make nano-beads move to a capture antibody, thus detecting an antigen with a small amount of blood within a short time.

While the present invention has been shown and described in connection with the exemplary embodiments, it will be apparent to those skilled in the art that modifications and variations can be made without departing from the spirit and scope of the invention as defined by the appended claims.

What is claimed is:

1. A method for detecting an antigen, the method comprising:
binding a first antibody and nano-beads to generate antibody nano-beads;
binding the generated antibody nano-beads and an antigen to generate antigen-antibody nano-beads;
forming at least one of an electric field and a magnetic field on the generated antigen-antibody nano-beads to bind the generated antigen-antibody nano-beads and a second antibody; and
detecting the antigen-antibody nano-beads bound to the second antibody.

2. The method of claim 1, wherein the nano-beads comprise at least one of a dielectric and a metal.

3. The method of claim 2, wherein the dielectric has a dipole moment.

4. The method of claim 1, wherein the nano-beads comprise a fluorescent component.

5. The method of claim 1, wherein, in generating the antigen-antibody nano-beads, at least one of an electric field and a magnetic field is formed on the antibody nano-beads.

6. The method of claim 1, wherein, in binding the generated antigen-antibody nano-beads and the second antibody, at least one of the electric field and the magnetic field is non-uniform.

7. The method of claim 1, wherein, in binding the generated antigen-antibody nano-beads and the second antibody, the second antibody is attached to a fixed position.

8. An apparatus for detecting an antigen, the apparatus comprising:
a mixing chamber for binding antibody nano-beads formed with a first antibody and nano-beads to an antigen to generate antigen-antibody nano-beads;
a detection chamber for binding the antigen-antibody nano-beads and a second antibody; and
an electromagnetic field generation unit for forming at least one of an electric field and a magnetic field on at least one of the mixing chamber and the detection chamber.

9. The apparatus of claim 8, wherein the nano-beads comprise at least one of a dielectric and a metal.

10. The apparatus of claim 9, wherein the dielectric has a dipole moment.

11. The apparatus of claim 8, wherein the nano-beads comprises a fluorescent component.

12. The apparatus of claim 8, wherein at least one of the electric field and the magnetic field of the electromagnetic field generation unit is non-uniform.

13. The apparatus of claim 8, wherein the second antibody is attached to the detection chamber.

14. A microfluidic chip comprising:
a mixing chamber for binding antibody nano-beads formed with a first antibody and nano-beads to an antigen to generate antigen-antibody nano-beads;
a detection chamber for binding the antigen-antibody nano-beads and a second antibody; and
an electromagnetic field generation unit for forming at least one of an electric field and a magnetic field on at least one of the mixing chamber and the detection chamber.

15. The microfluidic chip of claim 14, wherein the nano-beads comprise at least one of a dielectric and a metal.

16. The microfluidic chip of claim 15, wherein the dielectric has a dipole moment.

17. The microfluidic chip of claim 14, wherein the nano-beads comprise a fluorescent component.

18. The microfluidic chip of claim 14, wherein the second antibody is attached to the detection chamber.

19. The microfluidic chip of claim 14, wherein at least one of the electric field and the magnetic field of the electromagnetic field generation unit is non-uniform.

20. The microfluidic chip of claim 14, wherein the electromagnetic field generation unit is formed as at least one of an electrode array and a micro-coil array.
